SEQUENCE LISTING (21 sequences)

SEQ ID NO: 1 - DNA, 58 bp, Artificial Sequence
  adapter for library construction
aatgatacggcgaccaccgagatctacactctttccctacacgacgctcttccgatct

SEQ ID NO: 2 - DNA, 66 bp, Artificial Sequence
  adapter for library construction
agatcggaagagcacacgtctgaactccagtcacnnnnnnnnatctcgtatgccgtcttctgcttg

SEQ ID NO: 3 - DNA, 125 bp, Artificial Sequence
  library fragment flanked by adapter sequences
aatgatacggcgaccaccgagatctacactctttccctacacgacgctcttccgatctnagatcggaagagcacacgtctgaactccagtcacnnnnnnnnatctcgtatgccgtcttctgcttg

SEQ ID NO: 4 - DNA, 92 bp, Artificial Sequence
  adapter dimer
aatgatacggcgaccaccgagatctacactctttccctacacgacgctcttccgatctagatcggaagagcacacgtctgaactccagtcac

SEQ ID NO: 5 - DNA, 20 bp, Artificial Sequence
  unique sequence in adapter dimer
gacgctcttccgatctagat

SEQ ID NO: 6 - DNA, 53 bp, Artificial Sequence
  guide RNA shown with t in place of u
gaaacagctatgaccatgattacgccaagcacagtaatcgatttggagtttgg

SEQ ID NO: 7 - DNA, 53 bp, Artificial Sequence; identical to SEQ ID NO: 6
  guide RNA shown with t in place of u

SEQ ID NO: 8 - DNA, 237 bp, Artificial Sequence
  target sequence
agagagaccttggaaagcttcaatcaagattgtgcaatgctaagaattacgatggcgtttttgcattttccgatgataagacatatgtaattgctgatggcagcaatttgttccgacttgatggcacaaatgttgatgaaacatttgagccagtggagataaacgaagccttgaaaaacgcagattcaatgttttacgataaagttaataaaagactcgtcgtattcaaaggagaca

SEQ ID NO: 9 - DNA, 165 bp, Artificial Sequence
  non-target sequence
tcggtttgtacttgctgtaactttttttgtaattcttgcatctcttcatcttttttcaatttttctaattccttttctttcaattgctgttccaactgatctgcttcatccatggcgttttctttttccattttcatggacaacattttctttttaattgcttcc

SEQ ID NO: 10 - DNA, 20 bp, Artificial Sequence
  sequence targeted by guide RNA
agcaatttgttccgacttga

SEQ ID NO: 11 - DNA, 19 bp, Artificial Sequence
  sequence targeted by guide RNA
agtcatcgtacgaaaaacc

SEQ ID NO: 12 - DNA, 55 bp, Artificial Sequence
  forward primer
agagagaccttggaaagcttcaacactctttccctacacgacgctcttccgatct

SEQ ID NO: 13 - DNA, 53 bp, Artificial Sequence
  forward primer
agctgccctcttttcagtcgacactctttccctacacgacgctcttccgatct

SEQ ID NO: 14 - DNA, 57 bp, Artificial Sequence
  reverse primer
tgtctcctttgaatacgacgagtgtgactggagttcagacgtgtgctcttccgatct

SEQ ID NO: 15 - DNA, 62 bp, Artificial Sequence
  reverse primer
ggaagcaattaaaaagaaaatgttgtccgtgactggagttcagacgtgtgctcttccgatct

SEQ ID NO: 16 - DNA, 23 bp, Artificial Sequence
  forward primer
tggcgtttttgcattttccgatg

SEQ ID NO: 17 - DNA, 22 bp, Artificial Sequence
  forward primer
accggtaactgcaactaagcct

SEQ ID NO: 18 - DNA, 22 bp, Artificial Sequence
  reverse primer
ggcttcgtttatctccactggc

SEQ ID NO: 19 - DNA, 24 bp, Artificial Sequence
  reverse primer
tcttccacacttactcgttctgct

SEQ ID NO: 20 - DNA, 122 bp, Artificial Sequence
  targeting guide template
aaaaaagcaccgactcggtgccactttttcaagttgataacggactagccttattttaacttgctatttctagctctaaaactcaagtcggaacaaattgctccctatagtgagtcgtatta

SEQ ID NO: 21 - DNA, 122 bp, Artificial Sequence
  non-targeting guide template
aaaaaagcaccgactcggtgccactttttcaagttgataacggactagccttattttaacttgctatttctagctctaaaaccggtttttcgtacgatgactccctatagtgagtcgtatta

(19) United States
(12) Patent Application Publication
(10) Pub. No.: US 2016/0053304 A1
(43) Pub. Date: Feb. 25, 2016

(54) Methods Of Depleting Target Sequences Using CRISPR

(71) Applicant: Whitehead Institute for Biomedical Research, Cambridge, MA (US)

(72) Inventors: Omri Wurtzel, Somerville, MA (US); Samuel LoCascio, Boston, MA (US); Peter Reddien, Cambridge, MA (US)

(21) Appl. No.: 14/802,886

(22) Filed: Jul. 17, 2015

Related U.S. Application Data
(60) Provisional application No. 62/026,447, filed on Jul. 18, 2014.

(51) Int. Cl. C12Q 1/68

(52) U.S. Cl. CPC C12Q 1/6806

(57) ABSTRACT

Methods of depleting one or more target nucleic acid sequences using the Clustered Regularly Interspaced Short Palindromic Repeats (CRISPR) and CRISPR associated (Cas) proteins (CRISPR/Cas) system are disclosed. Kits and methods of producing a library comprising select mRNA sequences using the CRISPR/Cas system are also disclosed.

### RELATED APPLICATION

This Application claims the benefit of U.S. Provisional Application No. 62/026,447, filed on Jul. 18, 2014. The entire teachings of the above application are incorporated herein by reference.

### BACKGROUND OF THE INVENTION

DNA libraries (e.g., cDNA) can be created from the RNA (e.g., messenger RNA) in a cell or other source. For instance, mRNA is obtained by purifying and isolating it from the rest of other cellular RNAs (e.g., tRNA and rRNA). Known and currently used purification methods are costly, time-consuming, and at times, require the use of specialized lab equipment.

Thus, a need exists for improved and simplified methods of creating DNA libraries, methods for mRNA enrichment, and methods to deplete unwanted RNA or other unwanted nucleic acid in a sample.

### SUMMARY OF THE INVENTION

Described herein is the use of the Clustered Regularly Interspaced Short Palindromic Repeats (CRISPR) and CRISPR associated (Cas) proteins (CRISPR/Cas) system to enrich mRNA in a sample. Also described herein are methods of using the CRISPR/Cas system to deplete one or more nucleic acids in a sample by targeting (e.g., cleaving) one or more nucleic acid sequences, including unwanted nucleic acid sequences found in DNA and RNA libraries.

Accordingly, in one aspect, the invention is directed to a method of depleting one or more target nucleic acid sequences in a sample comprising the one or more target nucleic acid sequences and one or more non-target nucleic acid sequences wherein each of the target nucleic acid sequences and the non-target nucleic acid sequences comprise a 5′ adapter and a 3′ adapter. In one aspect, the target nucleic acid does not have a 5′ and 3′ adapter and the target DNA is cleaved after first strand DNA synthesis or after second strand DNA synthesis (e.g., using a target site) but before attachment of adapters, followed by attachment (e.g., ligation) of adapters (e.g., for use in serial analysis of gene expression (SAGE) and its derivatives (e.g., SuperSage, LongSage)). The method comprises contacting the sample with one or more ribonucleic acid (RNA) sequences wherein all or a portion of each RNA sequence is complementary to all or a portion of at least one target nucleic acid sequence (e.g., that may or may not be present) in the sample, a CRISPR associated (Cas) protein having nuclease activity, and a nucleic acid sequence that interacts with the Cas protein, thereby producing a combination. The combination is maintained under conditions in which the RNA sequences are allowed to hybridize to all or a portion of the target nucleic acid sequence to which each RNA sequence forms a complement thereby forming one or more base paired structures, and the one or more base paired structures and the nucleic acid sequence that interacts with the Cas protein direct the Cas protein to deplete each of the target nucleic acid sequences, thereby depleting the target nucleic acid in the sample.

In another aspect, the invention is directed to a method of producing a mRNA library. The method comprises contacting a sample comprising select mRNA to be retained (included) in the library (e.g., specified RNA molecules) and target nucleic acid sequences to be depleted (excluded, removed, minimized) from the library, wherein the select mRNA and target nucleic acid sequences each comprise a 5′ adapter and a 3′ adapter, with one or more ribonucleic acid (RNA) sequences wherein all or a portion of each RNA sequence is complementary to all or a portion of at least one target nucleic acid sequence in the sample, a CRISPR associated (Cas) protein having nuclease activity, and a nucleic acid sequence that interacts with the Cas protein, thereby producing a combination. In one aspect, the target nucleic acid does not have a 5′ and 3′ adapter and the target DNA is cleaved after first strand DNA synthesis or after second strand DNA synthesis (e.g., using a target site) but before attachment of adapters, followed by attachment (e.g., ligation) of adapters (e.g., for use in serial analysis of gene expression (SAGE) and its derivatives (e.g., SuperSage, LongSage)). The combination is maintained under conditions in which the RNA sequences are allowed to hybridize to all or the portion of the target nucleic acid sequence to which each RNA sequence forms a complement thereby forming one or more base paired structures, and the one or more base paired structures and the nucleic acid sequence that interacts with the Cas protein direct the Cas protein to deplete each of the target nucleic acid sequences, thereby producing a library comprising the select mRNA.

In another aspect, the invention is directed to a kit for producing a library of one or more non-target nucleic acid sequences from a sample. The kit comprises one or more ribonucleic acid (RNA) sequences, wherein all or a portion of each RNA sequence is complementary to all or a portion of at least one or more target nucleic acid sequences that may be present in the sample that are to be excluded from the library. The kit can also comprise a CRISPR associated (Cas) protein having nuclease activity, a nucleic acid sequence that interacts with the Cas protein, and/or one or more 5′ adapters and one or more 3′ adapters that can be used to bind (e.g., ligate, hybridize) to each of the one or more target nucleic acid sequences and each of the one or more non-target nucleic acid sequences in the sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic showing the approximate percentage of RNA types in a cellular RNA extract.

FIG. 2 is a schematic showing creation of a RNA-seq library for next-generation sequencing.

FIG. 3 is a schematic showing depletion of undesired sequences by CRISPR targeting using rRNA as an example.

FIG. 4 is a schematic showing removal of adapter concatamer (e.g., dimer) contamination from libraries.

FIG. 5 is a graph showing depletion of undesired sequences from a mixture of PCR products. No guide (#1, #2): representation of the targeted and non-targeted sequence in a reaction without any gRNA; Rep (#1, #2): depletion by incubating 20 μl reaction for 30 minutes, according to recommended Cas9 protocol by Manufacturer (NEB, #M0386L); ×2 Cas9 (#1, #2): increasing the volume of Cas9 in the reaction by two-fold over the recommended volume by the Cas9 Manufacturer (NEB, #M0386L); PEG (#1, #2): setting the reaction by replacing the H₂O in the reaction with 50% PEG8000; ×2 Time (#1, #2): extending the incubation to 2 hours; Complex population (#1, #2): replacing 50% of the reaction nucleic acid with yeast double stranded DNA. #1 and #2 represent duplicates of the same reaction condition.

### DETAILED DESCRIPTION OF THE INVENTION

A description of example embodiments of the invention follows.

Described herein is the development of an efficient technology for creating, enriching, and purifying nucleic acid sequences in a sample such as a library (e.g., DNA or RNA libraries). Specifically, the clustered regularly interspaced short palindromic repeats (CRISPR) and CRISPR associated genes (Cas genes), referred to herein as the CRISPR/Cas system, has been adapted as an efficient technology for enriching one or more nucleic acid sequences (e.g., mRNA) and/or for removing (deleting) other (e.g., undesired) nucleic acid sequences from a sample (e.g., a DNA library). Demonstrated herein is that the CRISPR/Cas system allows for the removal of one or more nucleic acid sequences targeted for depletion (targeted nucleic acids) in a sample.

Accordingly, in one aspect, the invention is directed to a method of depleting one or more target nucleic acid sequences in a sample comprising the one or more target nucleic acid sequences and one or more non-target nucleic acid sequences wherein each of the target nucleic acid sequences and the non-target nucleic acid sequences comprise a 5′ adapter and a 3′ adapter. The method comprises contacting the sample with one or more ribonucleic acid (RNA) sequences wherein all or a portion of each RNA sequence is complementary to all or a portion of at least one target nucleic acid sequence (e.g., that may or may not be present) in the sample, a CRISPR associated (Cas) protein having nuclease activity, and a nucleic acid sequence that interacts with the Cas protein, thereby producing a combination. The combination is maintained under conditions in which the RNA sequences are allowed to hybridize to all or a portion of the target nucleic acid sequence to which each RNA sequence forms a complement thereby forming one or more base paired structures, and the one or more base paired structures and the nucleic acid sequence that interacts with the Cas protein direct the Cas protein to deplete each of the target nucleic acid sequences, thereby depleting the target nucleic acid in the sample.

In another aspect, the invention is directed to a method of producing an mRNA library. The method comprises contacting a sample comprising select mRNA to be included in the library and target nucleic acid sequences to be excluded from the library, wherein the select mRNA and target nucleic acid sequences each comprise a 5′ adapter and a 3′ adapter, with one or more ribonucleic acid (RNA) sequences wherein all or a portion of each RNA sequence is complementary to all or a portion of at least one target nucleic acid sequence in the sample, a CRISPR associated (Cas) protein having nuclease activity, and a nucleic acid sequence that interacts with the Cas protein, thereby producing a combination. The combination is maintained under conditions in which the RNA sequences are allowed to hybridize to all or the portion of the target nucleic acid sequence to which each RNA sequence forms a complement thereby forming one or more base paired structures, and the one or more base paired structures and the nucleic acid sequence that interacts with the Cas protein direct the Cas protein to deplete each of the target nucleic acid sequences, thereby producing a library comprising the select mRNA. In a particular aspect, the select mRNA are in the form of DNA molecules derived from the select RNA. In one aspect, the target nucleic acid sequences that are cleaved are DNA copies of the target RNA (e.g., produced by reverse transcription of target RNA and, in at least some embodiments, second strand synthesis), and a library produced according to the methods comprises DNA (e.g., double stranded DNA) derived from (e.g., a copy of) the select RNA by reverse transcription of the RNA and synthesis of a second DNA strand complementary to the first strand. The afore-mentioned method may also be applied to produce libraries of other RNAs of interest, such as microRNAs.

As used herein “select mRNA” refers to mRNA to be included in the library. As will be appreciated by one of skill in the art, it may be desired to exclude (deplete, remove minimize) target nucleic acid sequences (e.g., rRNA, tRNA, certain mRNAs, adapter sequences introduced during library construction) in a library.

In yet another aspect, the invention is directed to a kit for producing a library of one or more non-target nucleic acid sequences from a sample. The kit comprises one or more ribonucleic acid (RNA) sequences, wherein all or a portion of each RNA sequence is complementary to all or a portion of at least one or more target nucleic acid sequence that is to be excluded from the library in the sample. The kit can also comprises a CRISPR associated (Cas) protein having nuclease activity. The kit can further comprise a nucleic acid sequence that interacts with the Cas protein. The kit can further comprises one or more 5′ adapters and one or more 3′ adapters that bind (e.g., ligate, hybridize) to each of the one or more target nucleic acid sequences and each of the one or more non-target nucleic acid sequences in the sample. In addition, the kit can further comprise components (e.g., reagents such as buffers, enzymes and the like) for nucleic acid isolation e.g., RNA or DNA isolation (extraction) from a sample.

As used herein, “deplete” or “depleting” one or more target nucleic acid sequences in a sample refers to complete or partial removal (deletion, elimination, minimization) of the one or more target nucleic acid sequences. The one or more target nucleic acid sequences can be depleted by cleaving, nicking or degrading all or a portion of the one or more target nucleic acids. For example, depleting one or more target nucleic acid sequences includes depleting one or more nucleotides (e.g., a portion of the target nucleic acid sequence; a substantial portion of a target nucleic acid sequence; the entire nucleic acid sequence) of the target nucleic acid sequence. In a particular aspect, depleting one or more target nucleic acid sequences refers to rendering the target nucleic acid sequences unavailable for amplification (e.g., exponential amplification, for example, the depleted target nucleic acid sequences cannot be amplified).

As will be apparent to those of skill in the art, a variety of nucleic acid sequences can be targeted for depletion. The target nucleic acid sequence can be a single stranded nucleic acid sequence and/or a double stranded nucleic acid sequence. The target nucleic acid can comprise DNA, RNA, or a combination thereof. The target nucleic acid sequences can be naturally occurring and/or synthetic nucleic acid sequences. Examples of RNA targeted for depleting include ribosomal RNA (rRNA), transfer RNA (tRNA), small RNA, small nucleolar RNA, messenger RNA (mRNA), signal recognition particle RNA (SRP RNA), transfer-messenger RNA (tmRNA), and mitochondrial RNA (mtRNA), and combinations thereof. In some aspects, the one or more target nucleic acid sequences comprise mRNA, rRNA, tRNA, mtRNA, or combinations thereof. Examples of DNA targeted for depletion include any RNA sequence targeted for depletion that has been reverse transcribed to generate complementary DNA (cDNA), repeat DNA sequence, transposon and mobile genetic elements sequences, adaptor sequence and combinations thereof. For example, in some embodiments, the one or more nucleic acid sequences targeted for depleting comprise cDNA that has been reverse transcribed from ribosomal RNA (rRNA), transfer RNA (tRNA), small RNA, small nucleolar RNA, messenger RNA (mRNA), signal recognition particle RNA (SRP RNA), transfer-messenger RNA (tmRNA), or mitochondrial RNA (mtRNA), and combinations thereof. Other types of DNA that can be targeted for depletion includes mitochondrial DNA (mtDNA), autosomal DNA, X chromosome DNA, Y chromosome DNA, plasmid DNA, viral DNA, phage DNA, and mobile genetic elements DNA. In some embodiments, prokaryotic rRNA is 5S, 16S, or 23S rRNA. In some embodiments, eukaryotic rRNA is 5S, 5.8S, 28S, or 18S rRNA.

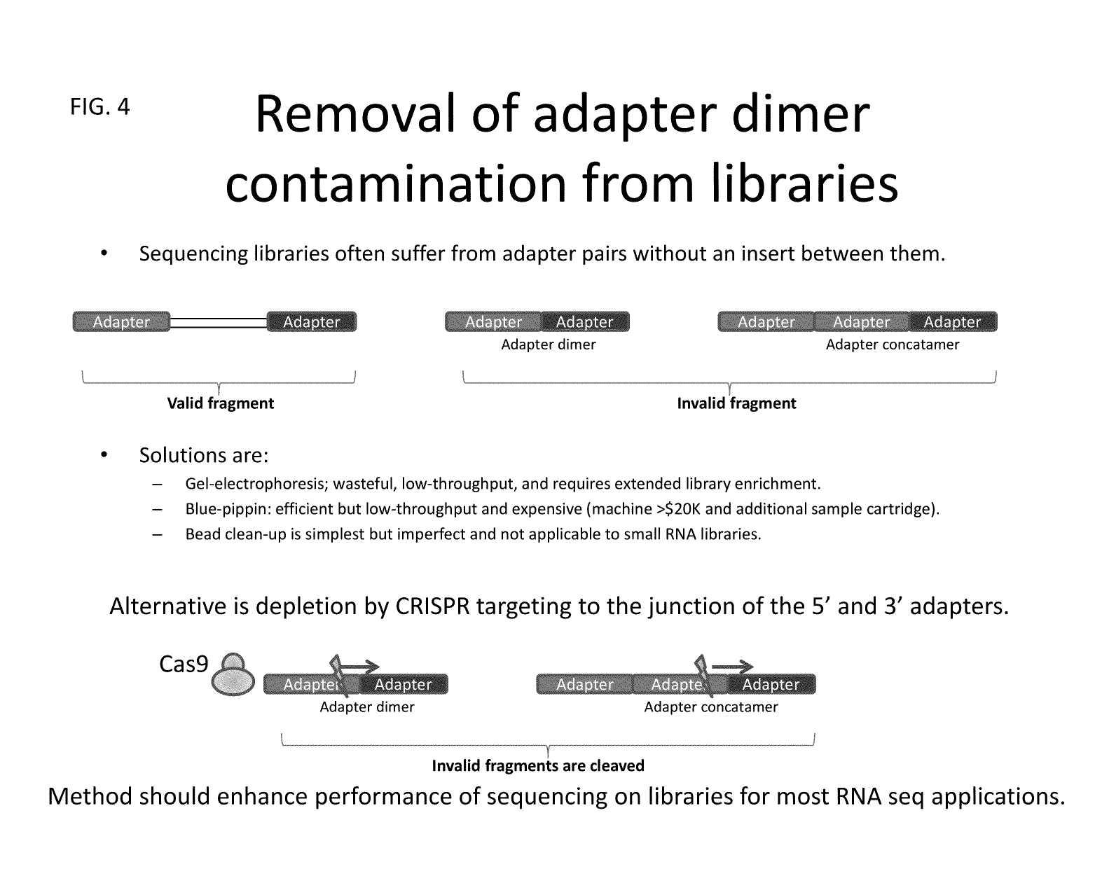

In one aspect, the target nucleic acid sequence is a contaminant. An example of contaminant nucleic acid sequences includes one or more adapter sequences. For example, as shown in FIG. 4, sequencing libraries often suffer from the presence of adapter pairs without an insert between them, e.g., resulting in the generation of adapter concatamers (e.g., adapter dimer, primer dimer) without an insert (e.g., intervening sequence between one or more adapter). The methods provided herein can be used to target these and other contaminating sequences, e.g., using one or more RNA sequences that are complementary to all or a portion of an adapter concatamer (e.g., an RNA sequence that is complementary to a (one or more) region at the junction of an adapter concatamer).

As will be apparent to one of skill in the art, the target nucleic acid can be a variety of lengths. For example, the target nucleic acid can be about 1 nucleotide, 2 nucleotides, 3 nucleotides, 4 nucleotides, 5 nucleotides, 10 nucleotides, 20 nucleotides, 30 nucleotides, 40 nucleotides, 50 nucleotides, 100 nucleotides, 200 nucleotides, 500 nucleotides, 1000 nucleotides, 2000 nucleotides or 5000 nucleotides. The target nucleic acid sequence can also be from about 1 nucleotide to about 5000 nucleotides, from about 2 nucleotides to about 2000 nucleotides, from about 3 nucleotides to about 1000 nucleotides, from about 4 nucleotides to about 500 nucleotides, from about 5 nucleotides to about 200 nucleotides, from about 10 nucleotides to about 100 nucleotides, or from about 20 nucleotides to about 50 nucleotides.

In some embodiments, a single target nucleic acid is targeted. In other aspects, more than one (multiple) target nucleic acid (e.g., 2, 3, 4, 5, 6, 7, 8, 9, 10, 20, 30, 40, 50, 60, 70, 80, 90, 100, 1000, 2000, 5000, 10000, 50000) is targeted. In some aspects, the targeted nucleic acid comprises all, or substantially all, of the nucleic acid in a sample excluding non-targeted nucleic acid.

In the methods provided herein, the one or more target nucleic acids in a sample is contacted with one or more ribonucleic acid (RNA) sequences that comprise a portion that is complementary to all or a portion of one or more target nucleic acid sequences. As used herein, the RNA sequence is sometimes referred to as guide RNA (gRNA) or single guide RNA (sgRNA). See, for example, U.S. Pat. Nos. 8,697,359 and 8,771,945 which are incorporated herein by reference.

In some aspects, the (one or more) RNA sequence can be complementary to one or more (e.g., some; all) of the one or more nucleic acids that are being targeted. In one aspect, the RNA sequence is complementary to all or a portion of a single target nucleic acid. In a particular aspect in which two or more target nucleic acid sequences are to be depleted, multiple (e.g., 2, 3, 4, 5, 6, 7, 8, 9, 10, or more than 10) RNA sequences can be introduced wherein each RNA sequence is complementary to, or specific for, all or a portion of at least one target nucleic acid sequence. In some aspects, two or more, three or more, four or more, five or more, or six or more, etc., RNA sequences are complementary to (specific for) different parts of a single target nucleic acid sequence. In other aspects, two or more, three or more, four or more, five or more, six or more, etc., RNA sequences are complementary to all or a portion of multiple target nucleic acid sequences (e.g., wherein some of the multiple RNA sequences are complementary to all or a portion of the same target nucleic acid sequence; wherein each of the multiple RNA sequences is complementary to all or a portion of a different (unique) target nucleic acid sequence or to a different (unique) region of a target nucleic acid sequence). In one aspect, two or more RNA sequences bind to different sequences (portions) of the same region (e.g. promoter) of a target nucleic acid sequence. In some aspects, a single RNA sequence is complementary to at least two or more (e.g., all) of the target nucleic acids. It will also be apparent to those of skill in the art that the RNA sequence that is complementary to one or more of the target nucleic acids and the sequence comprising a nucleic acid sequence that interacts with Cas protein can be introduced as a single sequence or as 2 (or more) separate sequences. It will also be apparent to those of skill in the art that the RNA sequence that is complementary to one or more of the target nucleic acids and the sequence comprising a nucleic acid sequence that interacts with Cas protein can be introduced as a single RNA molecule or as 2 (or more) separate RNA molecules. If the sequences are introduced as two (or more) separate RNA molecules, the hybridization of the RNA molecules results in a complex that serves to both hybridize to the target nucleic acid sequence and to recruit the Cas9 protein for cleavage.

In some aspects, the RNA sequence used to hybridize to a target nucleic acid is a naturally occurring RNA sequence, a modified RNA sequence (e.g., a RNA sequence comprising one or more modified bases), a synthetic RNA sequence, or a combination thereof. As used herein a “modified RNA” is an RNA comprising one or more modifications (e.g., RNA comprising one or more non-standard and/or non-naturally occurring bases) to the RNA sequence (e.g., modifications to the backbone and or sugar). Methods of modifying bases of RNA are well known in the art. Examples of such modified bases include those contained in the nucleosides 5-methylcytidine (5mC), pseudouridine (Ψ), 5-methyluridine, 2′O-methyluridine, 2-thiouridine, N-6 methyladenosine, hypoxanthine, dihydrouridine (D), inosine (I), and 7-methylguanosine (m7G). It should be noted that any number of bases in a RNA sequence can be substituted in various embodiments. It should further be understood that combinations of different modifications may be used.

In some aspects, the RNA sequence is a morpholino. Morpholinos are typically synthetic molecules, of about 25 bases in length and bind to complementary sequences of RNA by standard nucleic acid base-pairing. Morpholinos have standard nucleic acid bases, but those bases are bound to morpholine rings instead of deoxyribose rings and are linked through phosphorodiamidate groups instead of phosphates. Morpholinos do not degrade their target RNA molecules, unlike many antisense structural types (e.g., phosphorothioates, siRNA). Instead, morpholinos act by steric blocking and bind to a target sequence within a RNA and block molecules that might otherwise interact with the RNA.

Each of the one or more RNA sequences that comprises a portion that is complementary to all or a portion of one or more target nucleic acid sequences can vary in length from about 10 base pairs (bp) to about 200 bp. In some embodiments, the RNA sequence can be about 11 to about 190 bp; about 12 to about 150 bp; about 15 to about 120 bp; about 20 to about 100 bp; about 30 to about 90 bp; about 40 to about 80 bp; about 50 to about 70 bp in length.

The portion of each target nucleic acid sequence to which each RNA sequence is complementary can also vary in length. In particular aspects, the portion of each target nucleic acid sequence to which the RNA is complementary can be about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34,35, 36, 37, 38 39, 40, 41, 42, 43, 44, 45, 46 47, 48, 49, 50, 51, 52, 53,54, 55, 56,57, 58, 59 60, 61, 62, 63, 64, 65, 66, 67, 68, 69, 70, 71, 72, 73, 74, 75, 76, 77, 78, 79, 80 81, 82, 83, 84, 85, 86, 87 88, 89, 90, 81, 92, 93, 94, 95, 96, 97, 98, or 100 nucleotides (e.g., contiguous nucleotides; non-contiguous nucleotides) in length. In some embodiments, each RNA sequence can be at least about 70%, 75%, 80%, 85%, 90%, 95%, 98%, 99% 100%, etc. identical or similar to the portion of each target nucleic acid. In some embodiments, each RNA sequence is completely (fully) or partially complementary or similar to each target nucleic acid. For example, each RNA sequence can differ from perfect complementarity to the portion of the target nucleic acid by about 0, 1, 2, 3, 4, 5, 6, 7, 8, 9, 10, 11, 12, 13, 14, 15, 16, 17, 18, 19, 20, etc. nucleotides. In some embodiments, one or more RNA sequences are perfectly (fully) complementary (100%) across at least about 10 to about 25 (e.g., about 20) nucleotides of the target nucleic acid.

In the methods provided herein, the one or more target nucleic acids are contacted with a CRISPR associated (Cas) protein having nuclease activity (e.g., RNA guided (gRNA) nuclease activity). See, for example, U.S. Pat. Nos. 8,697,359 and 8,771,945 which are incorporated herein by reference. Bacteria and Archaea have evolved an RNA-based adaptive immune system that uses CRISPR (clustered regularly interspaced short palindromic repeat) and Cas (CRISPR-associated) proteins to detect and destroy invading viruses and plasmids (Horvath and Barrangou, Science, 327(5962):167-170 (2010); Wiedenheft et al., Nature, 482(7385):331-338 (2012)). Cas proteins, CRISPR RNAs (crRNAs) and trans-activating crRNA (tracrRNA) form ribonucleoprotein complexes, which target and degrade specific foreign nucleic acids, guided by crRNAs (Gasiunas et al., Proc. Natl. Acad. Sci, 109(39):E2579-86 (2012); Jinek et al., Science, 337:816-821 (2012)). The components of this system are used in the methods described herein and include a guide RNA (gRNA), a CRISPR associated nuclease (e.g., Cas9). The gRNA/Cas9 complex can be recruited to a target sequence by the base-pairing between the gRNA and the target sequence. Binding of Cas9 to the target sequence also requires the correct Protospacer Adjacent Motif (PAM) sequence adjacent to the target sequence. The binding of the gRNA/Cas9 complex localizes the Cas9 to the target nucleic acid sequence so that the Cas9 can cut both strands of nucleic acid (e.g., DNA).

In particular aspects in which the target nucleic acid sequence does not comprise a PAM sequence, the method can further comprise introducing one or more PAM sequences into the target nucleic acid sequence (e.g., when the target nucleic acid sequence is a contaminating sequence such as an adapter concatamer; before library construction).

In the methods provided herein, one or more Cas proteins or variants thereof cleave or nick each of the target nucleic acids. Any variant of Cas9 that retains RNA guided nuclease activity can be used in the methods of the invention. In some aspects, the binding of the gRNA/Cas9 complex localizes the Cas9 to the target nucleic acid so that the Cas9 can cut one strand or both strands of nucleic acid (e.g., DNA).

In some aspects, the invention is directed to the methods described herein, wherein the Cas protein is Cas9. In some aspects of the invention, the method of depleting one or more target nucleic acid sequences comprises introducing a Cas nucleic acid sequence or a variant thereof that encodes a Cas9 protein. In some aspects, the Cas nucleic acid sequence encodes a Cas9 protein that comprises one or more mutations.

The Cas protein can cleave one strand or both strands (e.g., of a double stranded target nucleic acid), or alternatively, nick one strand or both strands (e.g., of a double stranded target nucleic acid). In some aspects, a Cas9 nickase may be generated by inactivating one or more of the Cas9 nuclease domains. In some embodiments, an amino acid substitution at residue 10 in the RuvC I domain of Cas9 converts the nuclease into a DNA nickase. For example, the aspartate at amino acid residue 10 can be substituted for alanine (Cong et al., Science, 339:819-823). Other amino acid mutations that create a catalytically inactive Cas9 protein include mutating at residue 10 and/or residue 840. Mutations at both residue 10 and residue 840 can create a catalytically inactive Cas9 protein, sometimes referred to herein as dCas9. For example, a D10A and a H840A Cas9 mutant is catalytically inactive. In this aspect, depletion of desired sequences can be done by pull down of undesired fragments, e.g., a catalytically inactive Cas9 labeled with biotin could interact with a target nucleic acid through a gRNA and a tracrRNA, and instead of cutting the nucleic acid sequence, they are separated and eliminated by isolating the Cas9 with strepatavidin beads.

A variety of CRISPR associated (Cas) genes or proteins which are known in the art can be used in the methods of the invention and the choice of Cas protein will depend upon the particular conditions of the method (e.g., www.ncbi.nlm.nih.gov/gene/?term=cas9). Specific examples of Cas proteins include Cas1, Cas2, Cas3, Cas4, Cas5, Cas6, Cas7, Cas8, Cas9 and Cas10. In a particular aspect, the Cas nucleic acid or protein used in the methods is Cas9. In some embodiments a Cas protein, e.g., a Cas9 protein, may be from any of a variety of prokaryotic species. In some embodiments a particular Cas protein, e.g., a particular Cas9 protein, may be selected to recognize a particular protospacer-adjacent motif (PAM) sequence present in one or more of the target sequences. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes (Accession No. Q99ZW2), a S. thermophiles (Accession No. G3ECR1)), a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be used, e.g., to allow for recognition and modification of sites comprising the same, similar or different PAM motifs.

In the methods provided herein, the one or more target nucleic acids are contacted with a (one or more) nucleic acid sequence that interacts (complexes, binds) with a (one or more) Cas protein (a Cas interacting sequence). See, for example, U.S. Pat. Nos. 8,697,359 and 8,771,945 which are incorporated herein by reference. Nucleic acid sequences that interact with Cas protein and that along with based paired RNA structures direct Cas protein to deplete targeted sequences, are known in the art (e.g., see Jinek et al., Science, 337:816-821 (20123); Cong et al., Science, 339:819-823 (2013); Ran et al., Nature Protocols, 8(11):2281-2308 (2013); Mali et al., Sciencexpress, 1-5 (2013) all of which are incorporated herein by reference). In some aspects, such nucleic acid sequences are referred to as trans-activating CRISPR nucleic acid. In one aspect, the nucleic acid that interacts with Cas protein is an RNA sequence (sometimes referred to as trcrRNA). In other aspects, the nucleic acid sequence that interacts with a Cas protein can also hybridize to all or a portion of one or more of the RNA sequences that are complementary to all or a portion of at least one target sequence. In a particular aspect, the nucleic acid sequence that interacts with a Cas protein does not hybridize to all or the same portion of the RNA sequence that is complementary to all or a portion of at least one target sequence.

In one aspect, the one or more RNA sequences and the one or more nucleic acid sequences that interacts with the Cas protein are included as a single (the same) nucleic acid sequence. In another aspect, the nucleic acid sequence that interacts with the Cas protein is introduced as one or more separate nucleic acid sequences (e.g., not included in one, more or all of the one or more RNA sequences). In a particular aspect, upon hybridization of the one or more RNA sequences to the one or more target nucleic acids thereby forming one or more base paired structures, the one or more base paired structures and the nucleic acid sequence that interacts with the Cas protein direct the Cas protein or variants thereof to deplete the one or more target nucleic acids sequences.

After contacting the sample with the one or more RNA sequences that are complementary to all or a portion of at least one target nucleic acid sequence, the Cas protein and a nucleic acid sequence that interacts with the Cas protein to produce a combination and maintaining that combination under conditions in which the Cas protein depletes (cleaves, nicks, degrades) the target nucleic acid sequences, the target nucleic acid sequences no longer comprise both a 5′ adapter and a 3′ adapter by virtue of being cleaved or nicked by the Cas protein, whereas the non-target nucleic acid sequences do still have both a 5′ adapter and a 3′ adapter. Thus, the non-target nucleic acid sequences can now be separated or isolated from the target nucleic acid sequences for a variety of purposes (e.g., amplification (e.g., exponential amplification), cloning, sequencing, etc.).

The targeted sequences that no longer comprise both a 5′ adapter and a 3′ adapter by virtue of being depleted by the Cas protein, cannot be amplified (e.g., exponentially) using, e.g., a PCR, since PCR requires two primer sequences. Instead, the targeted sequences can be amplified linearly, and thus, will be negligible e.g., in a library. The targeted sequences that are linearly amplified will not be sequenced on commercially available sequencers (e.g., next-generation sequencers such as Illumina MiSeq® and/or HiSeq™, Applied Biosystems SOLiD™, Ion Torrent™) since they require two complete adapters for sequencing. In situations in which the library is used for cloning into a vector (e.g., Gateway reaction, a restriction enzyme based reaction), the targeted sequences will fail due to lack of compatible sequences on both ends of the target sequences.

In some aspects, the sample comprises the one or more target nucleic acid sequences and one or more non-target nucleic acid sequences. As will be apparent to one of skill in the art, the one or more non-target nucleic acid sequences comprises any nucleic acid sequence that is not targeted for depletion. In some aspects, the non-target nucleic acid sequences comprise single stranded nucleic acid sequence and/or double stranded nucleic acid sequences.

In some aspects, the non-target nucleic acid sequence in the sample is eukaryotic nucleic acid, prokaryotic nucleic acid, viral nucleic acid, synthetic nucleic acid, or modified nucleic acid.

In some aspects, the non-target nucleic acid in the sample is ribonucleic acid (RNA) or deoxyribonucleic acid (DNA). In some aspects, the RNA is mRNA or bacterial artificial chromosome. In some aspects, the DNA is cDNA or plasmid DNA.

Any of a variety of samples can be used in the methods of the invention. In some aspects, the sample is a library, a cell lysate, or a biological sample. In some aspects, the library is a DNA library, a RNA library, or an EST library. In some aspects, the biological sample is a fixed tissue sample, a sample of low-quality nucleic acids or a sample of degraded nucleic acids. In some aspects, the fixed tissue sample is a formalin fixed tissue sample. In some aspects, the biological sample is a frozen tissue sample. In some embodiments the tissue sample is a tumor sample. In some embodiments the tissue sample is from a tissue microarray. As will be appreciated by one of skill in the art, the sample can be prepared in a variety of ways.

In some aspects, the sample is from (e.g., derived from, taken from, obtained from) an organism. As will be appreciated by one of skill in the art, the sample can comprise one or more biological samples from an organism. In one aspect, the sample is from one or more cells, tissues, and/or extracts (e.g. lysates) thereof from the organism. In some aspects, the organism is a eukaryote or a prokaryote. In some aspects the eukaryote is an animal (e.g., human, mouse, rat, dog, cat, pig, chicken, cow, hamster, fish). In some aspects, the eukaryote is a plant. In some aspects, the prokaryote is a bacteria. In some aspects, the eukaryote is a fungus or invertebrate (e.g., an insect, a worm). In some aspects, the sample is from or comprises a pathogen (e.g., a parasite, pathogenic virus, pathogenic fungus, pathogenic bacterium, prion). In some embodiments the sample comprises one or more epithelial cells, endothelial cells, mesothelial cells, stem cells, germ cells, stem cells, immune system cells (e.g., T cell, B cell, dendritic cell, NK cell, macrophage, monocyte, granulocyte), fibroblasts, muscle cells, fat cells, nerve cells, gland cells, or mixtures thereof. In some embodiments a cell is a normal, healthy cell. In some embodiments a cell is a diseased cell or a cell suspected of being a diseased cell. In some aspects the sample is obtained from a tumor. In some aspects the sample is obtained from a primary tumor or from metastasis. In some embodiments the sample comprises one or more cancer cells. In some embodiments the sample is a biopsy sample, surgical sample, or body fluid sample or stool sample. A body fluid may be, e.g., blood, cerebrospinal fluid, exudate, pus, saliva, sputum, sweat, tears, urine. In some embodiments the sample is obtained or used to diagnose the presence or absence of a medical condition (e.g., a cancer, an infection by a pathogen), or to monitor a medical condition, evaluate its likelihood of recurrence, or its response to therapy, by depleting one or more target nucleic acid sequences in a sample and detecting the presence and/or abundance of particular nucleic acids remaining after depletion (e.g., by sequencing). In some embodiments, a library comprising non-target nucleic acids is generated from the sample as described herein. In some aspects, the sample is from (e.g., derived from, taken from, obtained from) the indoor or outdoor environment (e.g., the sample may be a soil, water (e.g., marine, fresh water, waste water), or air sample). In some aspects, the sample is from (e.g., derived from, taken from, obtained from) an inanimate object such as a wall, floor, machine, pipe, furniture, clothing, container, or the like, or a surface thereof.

The methods provided herein can further comprise isolating (non-target) nucleic acid sequences. As used herein, “isolated” nucleic acid sequence is substantially free from other components of the combination, e.g., pure; substantially pure, purified to homogeneity. Any of a variety of methods for isolation of (non-target) nucleic acid sequences can be used. Examples of such methods include (gel) electrophoresis, silica adsorption, alcohol (e.g., ethanol) precipitation, phenol-chloroform extraction, column chromatography, etc. Those of skill in the art will readily appreciate other methods of nucleic acid isolation e.g., RNA or DNA isolation (extraction) from a sample (e.g., fragmenting the mRNA or DNA copies thereof, end-repair, phosphorylation of the 5′ prime ends and/or A-tailing of the 3′ ends to facilitate ligation to sequencing adapters prior to adapter ligation, amplification before adapter ligation (e.g., in the case of small amounts of RNA, such as RNA from a single cell).

As provided herein, the one or more target nucleic acids and the one or more non-target nucleic acid sequences (e.g., mRNA to be included in a library) comprise a 5′ adapter and a 3′ adapter. As used herein, an “adapter” is a nucleic acid sequence that can be used to bind (e.g., ligate, hybridize) to a 5′ end and/or a 3′ end of one or more target nucleic acid sequences and/or one or more non-target nucleic acid sequences. As will be appreciated by those of skill in the art, a variety of 5′ and 3′ adapters can be used with the methods provided herein. Specific examples of adapters include:
AATGATACGGCGACCACCGAGATCTACACTCTTTCCCTACACGACGCTCTTC CGATCT (SEQ ID NO: 1) and AGATCGGAAGAGCACACGTCTGAACTCCAGTCACNNNNNNNNATCTCGTAT GCCGTCTTCTGCTTG (SEQ ID NO: 2) where N represents a barcode base on the adapter. A sequence in the library therefore has the following construct: 5′ AATGATACGGCGACCACCGAGATCTACACTCTTTCCCTACACGACGCTCTTC CGATCT—LIBRARY FRAGMENT—AGATCGGAAGAGCACACGTCTGAACTCCAGTCACNNNNNNNNATCTCGTAT GCCGTCTTCTGCTTG 3′ (SEQ ID NO: 3), where LIBRARY FRAGMENT is a particular nucleic acid sequence represented in the library. A primer dimer, which is a significant problem in a number of library construction protocols is manifested by having the adapters without an insert between them:
AATGATACGGCGACCACCGAGATCTACACTCTTTCCCTACACGACGCTCTTC CGATCTAGATCGGAAGAGCACACGTCTGAACTCCAGTCAC (SEQ ID NO: 4). A guide RNA designed against a sequence unique to the adapter dimer, which does not exist in library fragments that have an insert, such as GACGCTCTTCCGATCTAGAT (SEQ ID NO: 5), with the sequence CGG as the PAM, would effectively and specifically eliminate adapter dimers. This could be applied to other adapter sequences including those with other PAMs, by using a different Cas9 or a different adapter design.

The methods provided herein can further comprise amplifying (e.g., exponentially), sequencing and/or cloning nucleic acid sequences comprising a 5′ adapter and a 3′ adapter (e.g., non-target). Nucleic acid sequences comprising either a 5′ adapter or a 3′ adapter (nucleic acid sequences comprising only a 5′ adapter; nucleic acid sequences comprising only a 3′ adapter) are not exponentially amplified (e.g., target nucleic acid sequences that comprised a 5′ adapter and a 3′ adapter, but were cleaved by Cas9 in the method). As will be appreciated by those of skill in the art, exponential amplification methods (e.g., polymerase chain reaction (PCR)) require a 5′ adapter and a 3′ adapter on the sequence that is to be exponentially amplified. In addition, sequencing on next generation sequencers also require the sequence to have a 5′ adapter and a 3′ adapter, and thus, sequences that are amplified linearly would not be sequenced. In some aspects, all of the one or more non-target nucleic acids in the sample comprising a 3′ adapter and 5′ adapter are amplified. In other aspects, particular (selected) nucleic acid sequences are amplified.

Any of a variety of methods for amplification of (non-target) nucleic acid sequences can be used. Examples of such methods are polymerase chain reaction (PCR), ligase chain reaction (LCR), chain-termination methods, and sequence-specific isothermal amplification methods. In a particular aspect, the non-target nucleic acid is amplified using a polymerase chain reaction (PCR).

As will be appreciated by those of skill in the art, the length of the adapter sequence can vary. In some aspects, the adapter sequence is about 1 nucleotide to about 100 nucleotides in length. In some aspects, the adapter sequence is about 10 nucleotides to about 100 nucleotides in length. In other embodiments, the adapter sequence is about 5 nucleotides to about 80 nucleotides. In other embodiments, the adapter sequence is about 10 nucleotides to about 60 nucleotides. In other embodiments, the adapter sequence is about 15 nucleotides to about 40 nucleotides. In other embodiments, the adapter sequence is about 20 nucleotides to about 30 nucleotides. In some embodiments, the adapter sequence is less than 10 nucleotides (e.g., 1, 2, 3, 4, 5, 6, 7, 8, 9). In other embodiments, the adapter sequence is greater than 100 nucleotides.

As described herein, the one or more target nucleic acid to be depleted are contacted with one or more RNA sequences, a Cas protein, and a nucleic acid sequence that interacts with Cas protein thereby producing a combination. The combination is maintained under conditions in which the one or more RNA sequences hybridize to all or a portion of the one or more target nucleic acid sequences to which it forms a complement thereby forming one or more base paired structures, the one or more base paired structures and the nucleic acid sequence that interacts with Cas protein direct the Cas protein to deplete the one or more target nucleic acid sequences (e.g., by forming a complex (a CRISPR complex)), thereby depleting the target nucleic acid in the sample. See, for example, U.S. Pat. Nos. 8,697,359 and 8,771,945 which are incorporated herein by reference.

In some aspects of the invention, the method of depleting one or more target nucleic acids in a sample can comprise contacting the sample with the one or more RNA sequences, the Cas protein, and a nucleic acid sequence that interacts with Cas protein simultaneously. In another aspect, the method of depleting one or more target nucleic acids in a sample can comprise contacting the sample with the one or more RNA sequences, the Cas protein, and a nucleic acid sequence that interacts with Cas protein sequentially, e.g., in any order. As will be appreciated by one of skill in the art, the components of the combination and the methods described herein can be combined using known lab techniques and known solutions (e.g., buffers).

In some aspects, the invention is directed to the methods described herein, wherein the sample is maintained in an isothermal condition (e.g., at about 37° C.). In some aspects of the invention, the method of depleting one or more target nucleic acids comprises the combination being maintained or performed in an isothermal condition (e.g., at about 37° C.). In another aspect, the method of depleting one or more target nucleic acids comprises the combination being maintained or performed near isothermal conditions. In another aspect the combination is maintained or performed at a range of temperatures (e.g., about 0-100° C., about 4-10° C., about 37-95° C.) or at two or more different temperatures (e.g., at about 37° C. and then at about 50° C.) and a range of times (e.g., about 1 minute-60 minutes; about 1 hour-24 hours; about 36 hours to 48 hours, about 60 hours-a week or more). It will be appreciated by one of skill in the art which suitable or optimal temperature or temperatures are appropriate to maintain the combination.

The methods and compositions described herein can be used for a variety of purposes. For example, the methods and kits described herein can be used to deplete undesired sequences (e.g., rRNA, mtRNA) from RNA sequencing libraries made from single cells, which can be heavily contaminated with rRNA sequences (e.g, about 40-90%) as construction of single cell libraries is done by amplification of cDNA which generates double-stranded DNA that cannot be depleted by any available method.

The foregoing written specification is considered to be sufficient to enable one skilled in the art to practice the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments described herein, which fall within the scope of the claims. The scope of the present invention is not to be limited by or to embodiments or examples described above.

Section headings used herein are not to be construed as limiting in any way. It is expressly contemplated that subject matter presented under any section heading may be applicable to any aspect or embodiment described herein.

Embodiments or aspects herein may be directed to any agent, composition, article, kit, and/or method described herein. It is contemplated that any one or more embodiments or aspects can be freely combined with any one or more other embodiments or aspects whenever appropriate. For example, any combination of two or more agents, compositions, articles, kits, and/or methods that are not mutually inconsistent, is provided.

Articles such as “a”, “an”, “the” and the like, may mean one or more than one unless indicated to the contrary or otherwise evident from the context.

The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. Embodiments are provided in which exactly one member of the group is present, employed in, or otherwise relevant to a given product or process. Embodiments are provided in which more than one, or all of the group members are present, employed in, or otherwise relevant to a given product or process. Any one or more claims may be amended to explicitly exclude any embodiment, aspect, feature, element, or characteristic, or any combination thereof. Any one or more claims may be amended to exclude any agent, composition, amount, dose, administration route, cell type, target, cellular marker, antigen, targeting moiety, or combination thereof.

Embodiments in which any one or more limitations, elements, clauses, descriptive terms, etc., of any claim (or relevant description from elsewhere in the specification) is introduced into another claim are provided. For example, a claim that is dependent on another claim may be modified to include one or more elements or limitations found in any other claim that is dependent on the same base claim. It is expressly contemplated that any amendment to a genus or generic claim may be applied to any species of the genus or any species claim that incorporates or depends on the generic claim.

Where a claim recites a composition, methods of using the composition as disclosed herein are provided, and methods of making the composition according to any of the methods of making disclosed herein are provided. Where a claim recites a method, a composition for performing the method is provided. Where elements are presented as lists or groups, each subgroup is also disclosed. It should also be understood that, in general, where embodiments or aspects is/are referred to herein as comprising particular element(s), feature(s), agent(s), substance(s), step(s), etc., (or combinations thereof), certain embodiments or aspects may consist of, or consist essentially of, such element(s), feature(s), agent(s), substance(s), step(s), etc. (or combinations thereof). It should also be understood that, unless clearly indicated to the contrary, in any methods claimed herein that include more than one step or act, the order of the steps or acts of the method is not necessarily limited to the order in which the steps or acts of the method are recited.

Where ranges are given herein, embodiments in which the endpoints are included, embodiments in which both endpoints are excluded, and embodiments in which one endpoint is included and the other is excluded, are provided. It should be assumed that both endpoints are included unless indicated otherwise. Unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. “About” in reference to a numerical value generally refers to a range of values that fall within ±10%, in some embodiments ±5%, in some embodiments ±1%, in some embodiments ±0.5% of the value unless otherwise stated or otherwise evident from the context. In any embodiment in which a numerical value is prefaced by “about”, an embodiment in which the exact value is recited is provided. Where an embodiment in which a numerical value is not prefaced by “about” is provided, an embodiment in which the value is prefaced by “about” is also provided. Where a range is preceded by “about”, embodiments are provided in which “about” applies to the lower limit and to the upper limit of the range or to either the lower or the upper limit, unless the context clearly dictates otherwise. Where a phrase such as “at least”, “up to”, “no more than”, or similar phrases, precedes a series of numbers, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated.

Exemplification

### EXAMPLE 1

CRISPR-Based Targeting for Removal of Undesired Sequences from a Sample Containing a Mixture of Nucleic Acids

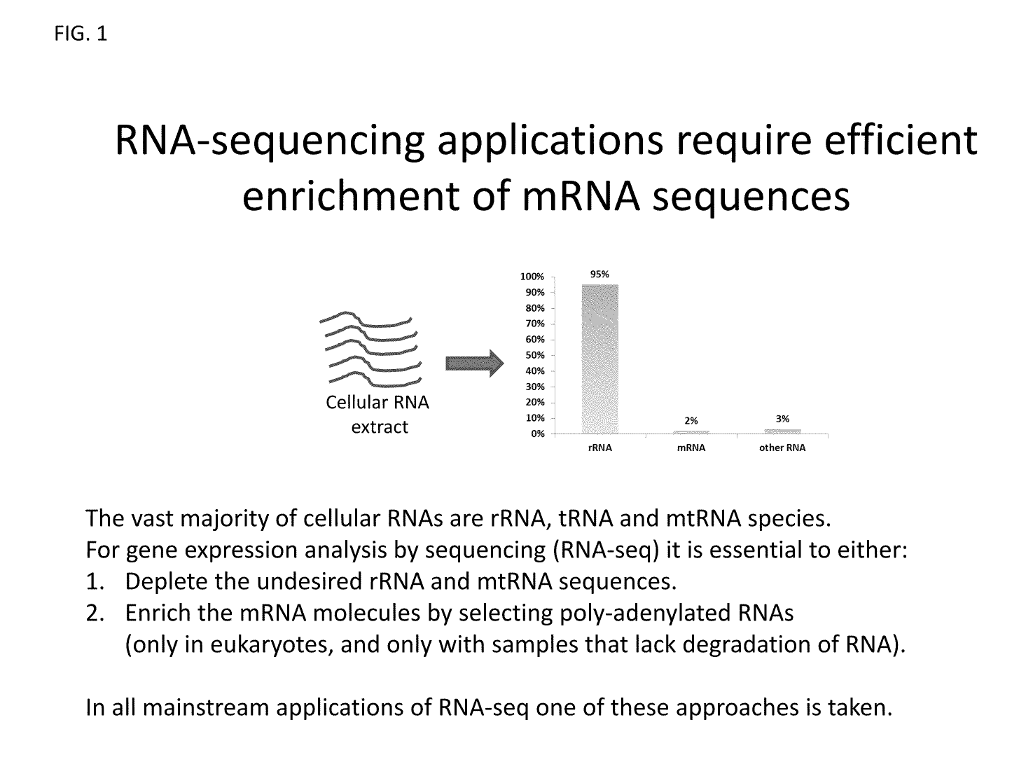

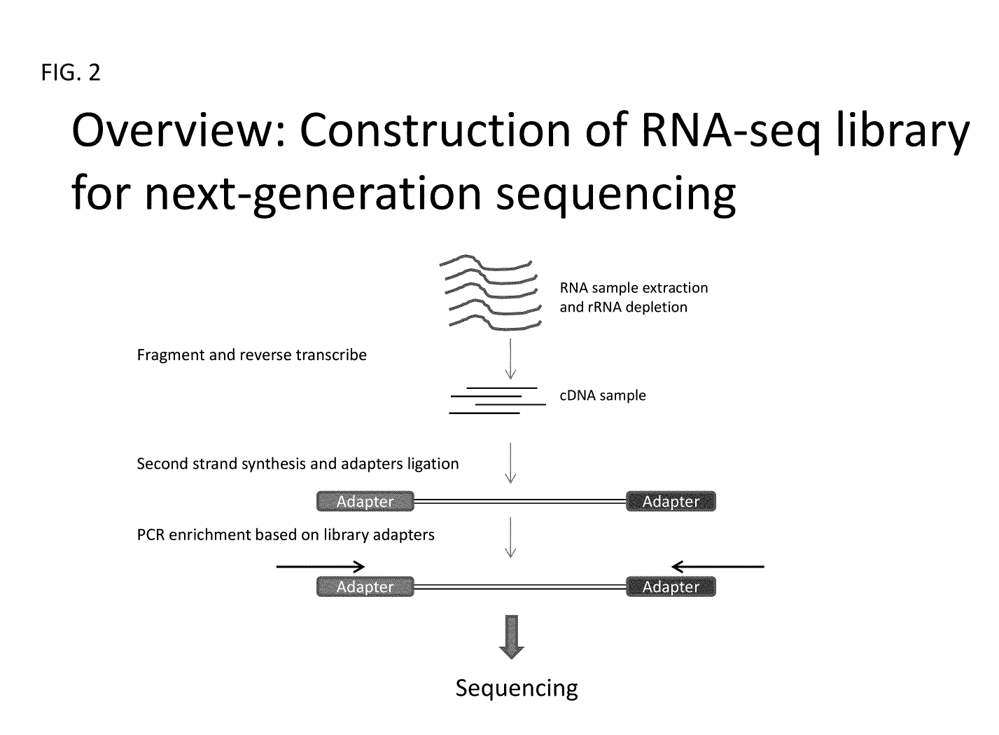

The vast majority of cellular RNA extract comprises unwanted nucleic acid material (e.g., rRNA, tRNA) shown in FIG. 1. Construction of a RNA-seq library using the methods described herein is outlined in FIG. 2 as an example of use of the methods provided herein. An RNA sample (e.g., cellular RNA extract of FIG. 1) is used and the rRNA in the sample is depleted by the methods described herein. The mRNA can be reverse transcribed into cDNA (complementary DNA). Library adapters (blue and red boxes) can be added to the 3′ and 5′ ends of the cDNAs. The cDNA can be enriched by PCR based on the library adapters. FIG. 2. provides an outline of library construction, without depletion of undesired sequences by the method described herein.

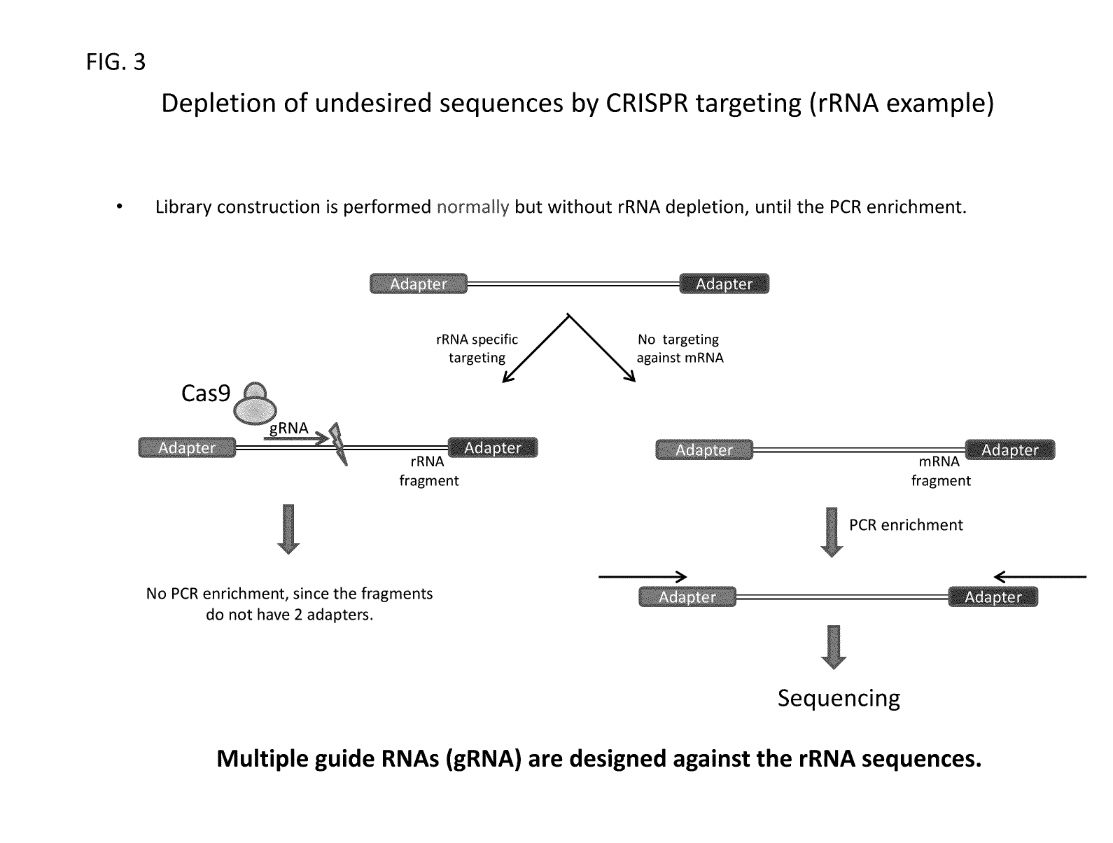

Specifically, depletion of undesired sequences (e.g., rRNA) by CRISPR/Cas targeting is shown in FIGS. 3 and 4. FIG. 3 shows one or more guide RNAs (gRNA; red arrow) specifically designed against the rRNA and other undesired sequences in a sample. CRISPR Cas interacts with nucleic acid sequences targeted by the guide RNA. A Cas protein, such as Cas9, can cleave all of the targeted nucleic acid sequences. Cleaved sequences are not enriched by PCR or other amplification methods, since the fragments do not have a 5′ and a 3′ adapter.

There are several advantages of using a CRISPR/Cas based method for depleting undesired or targeted nucleic acids in a sample. First, this system does not require polyA tails for enrichment. Using a CRISPR-based system allows for the depletion of any type of nucleic acid, such as RNA. Moreover, enrichment can occur quickly (e.g., 1 hour) and under isothermal conditions. Also, these methods work on double stranded (ds) DNA, avoiding the procedural risk involved with using RNA at room temperature (or higher). The methods described herein apply to any organism (e.g., eukaryote, prokaryote) or synthetic undesired sequences (e.g., primer dimers) and does not require the use of any special instruments (e.g., a high-powered magnet). These methods can work with RNA from any source (e.g., tissue samples, fixed tissue samples, clinical samples, etc.), including from samples in which polyA selection is not possible or difficult. Finally, one or more sets of guide RNAs can be designed based on the methods described herein. For example, one or more sets of guide RNA can be species-specific or organism-specific. A kit can comprise one or more gRNA sets. The kit may further comprise a Cas protein (e.g., Cas9) and other reaction components (e.g., reaction buffer).

The methods described herein can also remove or deplete adapter dimer contamination (see FIG. 4) from libraries. Many sequencing libraries often suffer from adapter pairs without an insert between them. Removal of adapter dimers is challenging. Currently solutions include: (i) gel electrophoresis, but is wasteful, low-throughput and requires extended library enrichment; (ii) BluePippin™ (Sage Science) is efficient, but is low-throughput and expensive (over $20,000 for the machine and additional costs for each sample cartridge or cassettes) and; (iii) bead clean-up is simple, but imperfect (e.g., primer dimer contamination is reduced but not eliminated) and not applicable to small RNA libraries (e.g., microRNA).

The methods described herein can be also used to remove or deplete adapter dimers, concatamers or other unwanted adapter combinations in a sample (FIG. 4). FIG. 4 also shows the removal and depletion of adapter dimers and concatamers by one or more guide RNAs (red arrows) specifically designed to target at or near the junction of the 5′ adapter and 3′ adapter. These invalid fragments are cleaved by CRISPR/Cas.

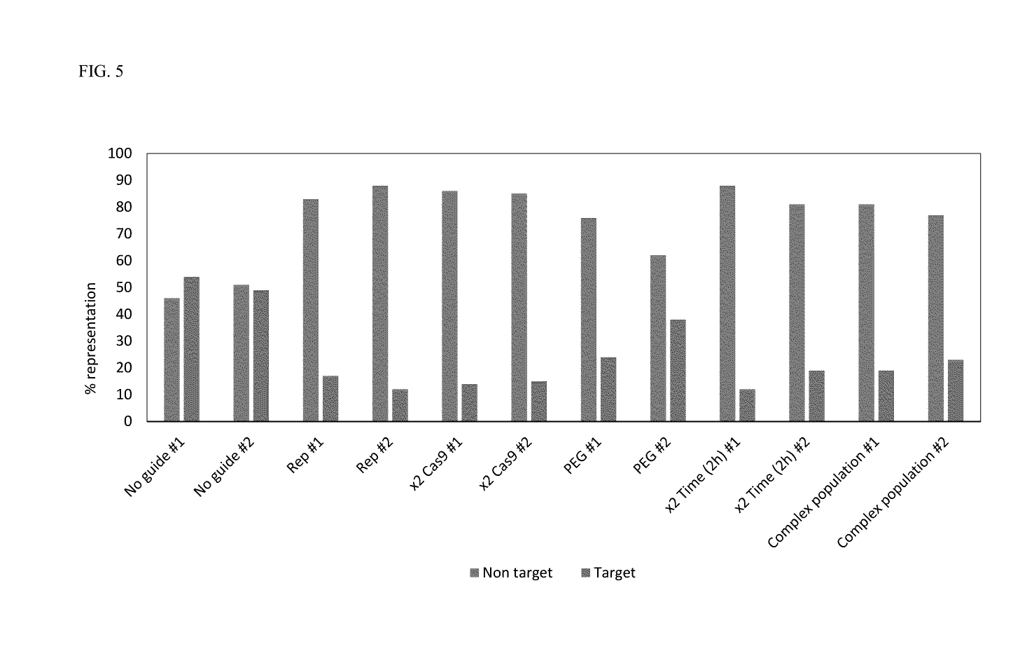

The efficacy of the present methods is illustrated in FIG. 5, which demonstrates that the methods described herein can remove or deplete undesired sequences from a sample containing a mixture of polymerase chain reaction (PCR) products. The Target (SEQ ID NO: 8) and Non-target (SEQ ID NO:9) sequences were inserted into a plasmid and amplified in separate reactions using the appropriate forward and reverse primers (“primer for amplification of insert” in Table 1). The purified products of the reactions were combined in equimolar ratios and undesired sequence (Target—SEQ ID NO: 8) was depleted by incubating Cas9 with gRNA. Briefly, gRNA against the PCR product of the Target sequence, or gRNA designed to target a sequence not found in the Target or Non-target sequence (i.e., the control gRNA) was incubated for 60 minutes. The representation of the Target was compared between the reactions by qPCR of the Target and Non target sequences in both reactions, and calculated by the AACT method, wherein the result from the control gRNA reaction was used for normalization. Results are shown in FIG. 5. The sequences of the gRNA, insert or qPCR primers, and target regions of the Target and Non-target are summarized in Table 1.

TABLE 1
Summary of sequences

| | Target | Non target |
|---|---|---|
| gRNA | GAAACAGCTATGACCATGATT | A gRNA to the Non-target |
| | ACGCCAAGCACAGTAATCGA | sequence was not designed |
| | TTTGGAGTTTGG (SEQ ID NO: | |
| | 6) | |
| Full insert | AGAGAGACCTTGGAAAGCTT | TCGGTTTGTACTTGCTGTAAC |
| sequence | CAATCAAGATTGTGCAATGC | TTTTTTTGTAATTCTTGCATC |
| | TAAGAATTACGATGGCGTTT | TCTTCATCTTTTTTCAATTTT |
| | TTGCATTTTCCGATGATAAG | TCTAATTCCTTTTCTTTCAAT |
| | ACATATGTAATTGCTGATGG | TGCTGTTCCAACTGATCTGCT |
| | CAGCAATTTGTTCCGACTTG | TCATCCATGGCGTTTTCTTTT |
| | ATGGCACAAATGTTGATGAA | TCCATTTTCATGGACAACAT |
| | ACATTTGAGCCAGTGGAGAT | TTTCTTTTTAATTGCTTCC |
| | AAACGAAGCCTTGAAAAACG | (SEQ ID NO: 9) |
| | CAGATTCAATGTTTTACGAT | |
| | AAAGTTAATAAAAGACTCGT | |
| | CGTATTCAAAGGAGACA | |
| | (SEQ ID NO: 8) | |
| | Underlined region indicates region | |
| | targeted by gRNA (see below, | |
| | SEQ ID NO: 10) | |
| Sequence | AGCAATTTGTTCCGACTTGA | A gRNA to the Non-target |
| targeted by | (SEQ ID NO: 10) | sequence was not designed |
| gRNA | | |
| forward PCR | AGAGAGACCTTGGAAAGCTT | AGCTGCCCTCTTTTCAGTCG |
| primer for | CAACACTCTTTCCCTACACG | ACACTCTTTCCCTACACGAC |
| amplification | ACGCTCTTCCGATCT (SEQ ID | GCTCTTCCGATCT (SEQ ID |
| of insert | NO: 12) | NO: 13) |
| reverse PCR | TGTCTCCTTTGAATACGACG | GGAAGCAATTAAAAAGAAA |
| primer for | AGTGTGACTGGAGTTCAGAC | ATGTTGTCCGTGACTGGAGT |
| amplification | GTGTGCTCTTCCGATCT (SEQ | TCAGACGTGTGCTCTTCCGA |
| of insert | ID NO: 14) | TCT (SEQ ID NO: 15) |
| qPCR | TGGCGTTTTTGCATTTTCCGA | ACCGGTAACTGCAACTAAGC |
| forward | TG (SEQ ID NO: 16) | CT (SEQ ID NO: 17) |
| primer | | |
| qPCR reverse | GGCTTCGTTTATCTCCACTGG | TCTTCCACACTTACTCGTTCT |
| primer | C (SEQ ID NO: 18) | GCT (SEQ ID NO: 19) |
| Guide | AAAAAAGCACCGACTCGGTG | A gRNA to the Non-target |
| template | CCACTTTTTCAAGTTGATAAC | sequence was not designed |
| | GGACTAGCCTTATTTTAACTT | |
| | GCTATTTCTAGCTCTAAAACT | |
| | CAAGTCGGAACAAATTGCTC | |
| | CCTATAGTGAGTCGTATTA | |
| | (SEQ ID NO: 20) | |

The gRNA that targets the Target sequence was synthesized from the single-stranded DNA template (“guide template”—SEQ ID NO: 20 in Table 1) using T7 polymerase. The control gRNA (the gRNA that does not target either the Target or Non-target sequence) had the sequence GAAACAGCTATGACCATGATTACGCCAAGCGGGTATGGAGTTCGTGAGGC (SEQ ID NO: 7), which was designed to target a sequence that contains a region having the sequence AGTCATCGTACGAAAAACC (SEQ ID NO: 11). The control gRNA was synthesized from the single-stranded DNA template AAAAAAGCACCGACTCGGTGCCACTTTTTCAAGTTGATAACGGACTAGCCTT ATTTTAACTTGCTATTTCTAGCTCTAAAACCGGTTTTTCGTACGATGACTCCC TATAGTGAGTCGTATTA (SEQ ID NO: 21).

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of depleting one or more target nucleic acid sequences in a sample comprising the one or more target nucleic acid sequences and one or more non-target nucleic acid sequences, wherein each of the target nucleic acid sequences and the non-target nucleic acid sequences comprise a 5′ adapter and a 3′ adapter comprising:
(a) contacting the sample with:
i) one or more ribonucleic acid (RNA) sequences wherein all or a portion of each RNA sequence is complementary to all or a portion of at least one target nucleic acid sequence in the sample;
ii) a CRISPR associated (Cas) protein having nuclease activity; and
iii) a nucleic acid sequence that interacts with the Cas protein;
thereby producing a combination; and
(b) maintaining the combination under conditions in which the RNA sequences are allowed to hybridize to all or a portion of the target nucleic acid sequence to which each RNA sequence forms a complement thereby forming one or more base paired structures, and the one or more base-paired structures and the nucleic acid sequence that interacts with the Cas protein direct the Cas protein to deplete each of the target nucleic acid sequences;
thereby depleting the target nucleic acid in the sample.

2. The method of claim 1, further comprising isolating the one or more non-target nucleic acid sequences from the sample.

3. The method of claim 1, further comprising amplifying the non-target nucleic acid sequences in the sample.

4. (canceled)

5. The method of claim 1, wherein the Cas protein is Cas9.

6. The method of claim 1, wherein the RNA sequence is from about 10 base pairs to about 200 base pairs in length.

7. (canceled)

8. (canceled)

9. The method of claim 1, wherein the non-target nucleic acid in the sample is ribonucleic acid (RNA) or deoxyribonucleic acid (DNA).

10. (canceled)

11. (canceled)

12. The method of claim 1, wherein the sample is a library, a cell lysate, or a biological sample.

13-15. (canceled)

16. The method of claim 1, wherein the one or more target nucleic acid sequences comprises deoxyribonucleic acid (DNA), ribonucleic acid (RNA), or a combination thereof.

17. (canceled)

18. (canceled)

19. The method of claim 1, wherein the sample is contacted with the one or more RNA sequences, the Cas protein, and the nucleic acid sequence that interacts with Cas protein simultaneously or sequentially.

20. (canceled)

21. A method of producing a mRNA library comprising:
(a) contacting a sample, wherein the sample comprises select mRNA to be included in the library and target nucleic acid sequences to be depleted from the library, and the select mRNA and target nucleic acid sequences each comprise a 5′ adapter and a 3′ adapter, with:
i) one or more ribonucleic acid (RNA) sequences wherein all or a portion of each RNA sequence is complementary to all or a portion of at least one target nucleic acid sequence in the sample;
ii) a CRISPR associated (Cas) protein having nuclease activity; and
iii) a nucleic acid sequence that interacts with the Cas protein;
thereby producing a combination;
(b) maintaining the combination under conditions in which the RNA sequences are allowed to hybridize to all or the portion of the target nucleic acid sequence to which each RNA sequence forms a complement thereby forming one or more base paired structures, and the one or more base paired structures and the nucleic acid sequence that interacts with the Cas protein direct the Cas protein to deplete each of the target nucleic acid sequences;
thereby producing a mRNA library comprising the select mRNA.

22. The method of claim 21, further comprising isolating the one or more non-target nucleic acid sequences from the sample.

23. The method of claim 21, further comprising amplifying the select mRNA in the sample.

24. (canceled)

25. The method of claim 21, wherein the Cas protein is Cas9.

26. The method of claim 21, wherein the RNA sequence is from about 10 base pairs to about 200 base pairs in length.

27. (canceled)

28. The method of claim 21, wherein the one or more target nucleic acid sequences comprises deoxyribonucleic acid (DNA), ribonucleic acid (RNA), or combinations thereof.

29-32. (canceled)

33. A kit for producing a library of one or more non-target nucleic acid sequences from a sample comprising:
one or more ribonucleic acid (RNA) sequences wherein all or a portion of each RNA sequence is complementary to all or a portion of at least one or more target nucleic acid sequence in the sample that is to be excluded from the library;
a CRISPR associated (Cas) protein having nuclease activity;
a nucleic acid sequence that interacts with the Cas protein; and
one or more 5′ adapters and one or more 3′ adapters that bind to each of the one or more target nucleic acid sequences and each of the one or more non-target nucleic acid sequences in the sample.

34. The kit of claim 33, wherein the RNA sequence and the nucleic acid sequence that interacts with Cas protein are on the same sequence.

35. The kit of claim 33, wherein the RNA sequence is from about 10 base pairs to about 200 base pairs in length.

36. The kit of claim 33, wherein the sample comprises a virus.

37. The kit of claim 33, wherein the sample comprises one or more cells from an organism.

38-40. (canceled)

41. The kit of claim 33, wherein the Cas protein is Cas9.

42. The kit of claim 33, further comprising one or more components for an amplification reaction.

43. The kit of claim 33, wherein the library is a mRNA library.
